(19) United States
(12) Patent Application Publication
(10) Pub. No.: US 2005/0126579 A1
(43) Pub. Date: Jun. 16, 2005

(54) Theory of bell's palsy

(76) Inventor: Anuthep Benja-Athon, New York, NY (US)

Correspondence Address: ANUTHEP BENJA-ATHON, M.D.;Pain Management, Acupuncture Physical Therapy, 210 E 36th St., New York, NY, 10016, US

(21) Appl. No.: 10/734,897

(22) Filed: Dec. 15, 2003

(51) Int. Cl. A61B 5/00; A01N 25/00; A61K 47/00; A61B 19/00

(52) U.S. Cl. 128/898; 514/789

(57) ABSTRACT

The Theory of Bell's Palsy derived from scientific evidences states that there are two types of facial paralysis based on two distinct inciting etiologies wherein a first facial paralysis is caused by a first viral, bacterial, spirochetal or fungal infection, a second facial paralysis is caused by a second vascular etiology associates with cardiovascular, endocrinologic, rheumatolgic diseases, trauma or any combination thereof. The second facial paralysis associates with vascular etiology, in general, is more virulent than said first facial paralysis with tendency towards poorer outcomes. Mathematic equations and graphs are also derived. It further provides a process of using medications for treating the inciting infectious, cardiovascular, endocrinologic, rheumatologic and trauma causes.

### FIELD OF INVENTION

The Theory of Bell's Palsy leading to a correct treatment of Bell's palsy.

### BACKGROUND OF THE INVENTION

The objectives of the present invention is the Theory of Bell's Palsy—a discovery which removes the cloak of 182-year mystery and misunderstanding shrouding and eliminates the medical malpractices on this common affliction of the facial nerve. The Theory further provides a correct and standized medical care for Bell's palsy benefiting about 64,000 Bell's palsy patients every year in America.

Since its first description by Sir Charles Bell in 1821, Bell's palsy permanently branded about 55%-90% afflicted victims with the life-long and painful facial deformity and disorders. Every 13 minutes one American was afflicted by Bell's palsy. (Adour, K. K. 1972) This potentially facial-disfiguring disease of epic proportion for a person has an incidence rate of about 23 per 100,000 people per year. (Hauser W. A. 1971). This figure translates to a staggering number of about 64,000 Americans per year.

Like stroke, Bell's palsy struck people in their prime of life and all ages and genders were not spared. A person might get up one morning and usually find that one side of the face was paralyzed. The paralysis is associate with other dysfunction of the face and mouth. A catastrophe resulting in utmost physical challenges to the victim. “A willing mind is imprisoned in an unwilling body™”, the paralyzed face may not only cause eternal disfigurement and psychological pain but also many disorders of and problems with sensations, taste, mastication and eating, vision and hearing. On the paralyzed face, the patient could neither smile nor whistle, had abnormal tearing, eyelids which failed to close causing injuries and damage to the eye, deranged taste, food trapped in the cheek, ringing in the ear and hearing disturbances, etc. Overnight, a career and livelihood were left in ruin or destroyed.

In prior art, Bell's palsy is defined as the paralysis or weakness of the unilateral or bilateral facial muscles caused by injury to the facial nerve of unknown cause. There were earlier reports showing an association between Bell's palsy and infections and chronic diseases. Uri Leibowitz 1966 thought that “the possibility is suggested that Bell's palsy may be the clinical manifestation of two different diseases processes: an infections-inflammatory process which is more common in cold weather and in young people and a vascular-ischemic process which is more frequent in warm weather and in the older age groups.” Neither proof nor research has ever been provided or done to advance the above thought. Consequently, physicians can offer only confusions to patients and their treatments, heretofore, were haphazard, incorrect and ineffective.

However, for the first time in the 182-year history of Bell's palsy, this patent applicant has accomplished extensive research providing the concrete proof and evidence leading to the present Theory of Bell's Palsy infra—Bell's palsy henceforth is the paralysis or weakness of the unilateral or bilateral facial muscles with infra causes. Uniquely, the Theory definitively claims a causal relationship between the compromise of the facial nerve and, first, viral, bacterial and spirachetal infections and, second, cardiovascular, endocrinologic, rheumatologic and trauma etiologies. Henceforth, Bell's palsy is also defined as the compression of the facial nerve and/or facial paralysis caused either by an infection, cardiovascular, endocrinologic, rheumatologic or trauma etiologies and any combination thereof leading to the compromise of the facial nerve.

### SUMMARY OF THE INVENTION

Prior to the present invention, physicians and laypeople are misinformed about Bell's palsy. These people are totally handicapped and lack a means for dealing with and treating Bell's palsy. This present invention contributes a solution to this 182-year problem and dilemma.

The Theory of Bell's Palsy uniquely derived from scientific evidences and data states that there are two types of facial paralysis based on two distinct inciting etiologies wherein a first facial paralysis is caused by a first viral, bacterial, spirochetal or fungal infection, a second facial paralysis is caused by a second vascular etiology associates with cardiovascular, endocrinologic, rheumatolgic diseases, trauma or any combination thereof. The second facial paralysis associates with vascular etiology, in general, is more virulent than said first facial paralysis with tendency towards poorer outcomes. Mathematic equations and graphs are also derived to explain supra. It further provides a process of correctly and appropriately using medications for treating the inciting infectious, cardiovascular, endocrinologic, rheumatologic and trauma causes of Bell's palsy.

### BRIEF DESCRIPTION OF THE DRAWINGS

FIG. 1—Bell's Curve

FIG. 2—Fair Bell's

### DESCRIPTION OF THE PREFERRED EMBODIMENTS

The Theory of Bell's Palsy™ thence a standard for the treatment and management of Bell's palsy are derived from the extensive research of this patent applicant. According to the Theory of Bell's Palsy™, facial paralysis is preceded by the abnormal, altered or decreased sensations and activities of the structures of the face and mouth.

Theory of Bell's Palsy™: Based on the scientific evidences derived from the research supra, the Theory uniquely and affirmatively shows that there are two distinct types of facial paralysis based on two distinct inciting etiologies. In the first type of Bell's palsy, facial paralysis is caused by a viral, bacterial, spirochetal or fungal infection. In the second type of Bell's palsy, facial paralysis is caused by a vascular etiology associates with cardiovascular, endocrinologic, rheumatolgic diseases or trauma. However, the common denominator for both types of Bell's palsy is the ensueing compression and ischemia of the facial nerve. Said first type occurs in all age groups, however, said second type is more common in older patients. Generally, the facial paralysis associates with vascular etiology is more virulent than said first facial paralysis and has the tendency towards poorer outcomes than said first facial paralysis.

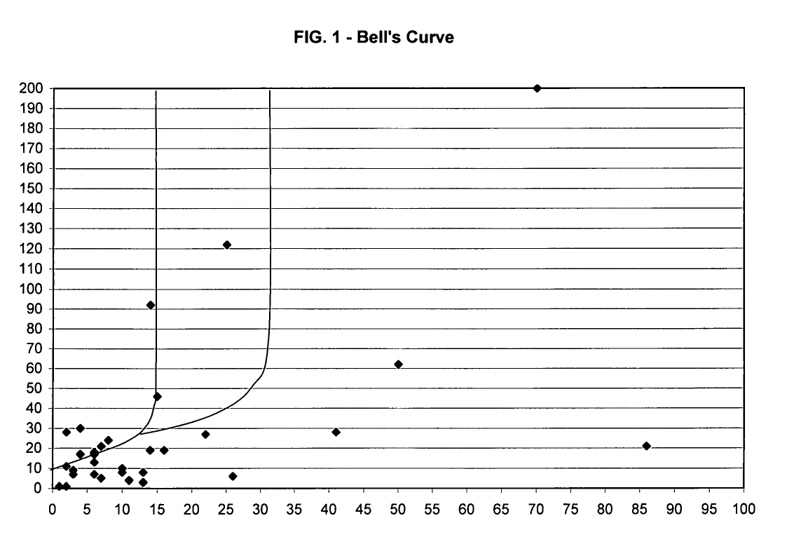

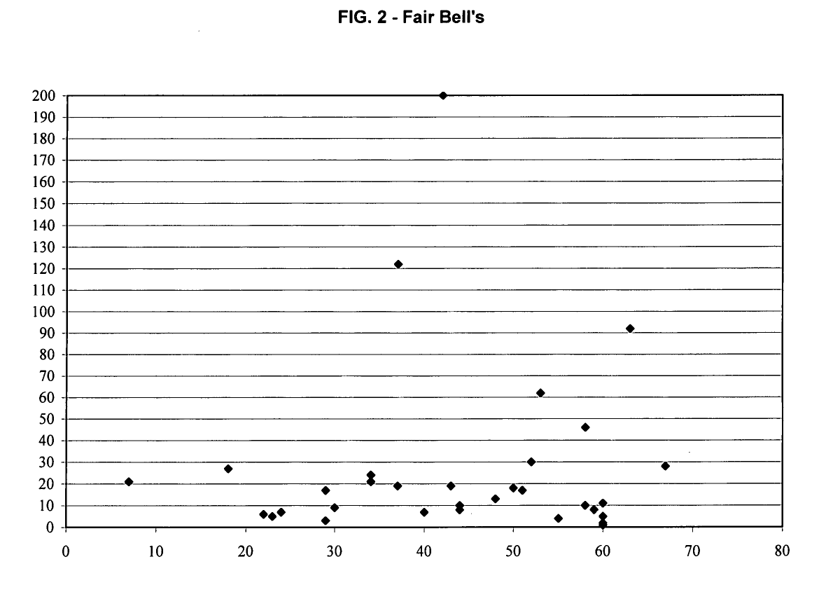

A mathematic means for defining the duration of the facial paralysis and recovery time from the facial paralysis is shown in FIG. 1—the Bell's Curve™. The relationship between the duration of the facial paralysis and recovery time from the facial paralysis is expressed as Y=f(X)+K. Specifically, said equation can be further expressed as Y=cA+cB+K. Another mathematic means for defining the recovery time from the facial paralysis and ages of patients is shown in FIG. 2—the Fair Bell's™.

The meaning of these mathematical equations and graphs expressed supra is understood by one skill in the art. Other mathematical equations and graphs can be derived and extrapolated from the evidences and data of the present invention. The Theory of Bell's palsy according to claim 1 wherein a mathematic means for defining the duration of the facial paralysis and recovery time from the facial paralysis is a plurality of graphs which can be further manipulated to yield other graphs expressing said relationships and phenomema.

Uniquely, the Theory advances the understanding of Bell's palsy as briefly explained supra by physicians and laypeople. Consequently, an effective and expeditious method and process of managing the etiologies facial paralysis comprise the use of a drug means for treating the inciting cardiovascular cause such as, but not limited to, a drug means for treating hypertension, artherosclerotic diseases and hyperlipidemia such as, but not limited to, diuretics, adrenergic blockers, adrenergic stimulants, alpha and beta adrenergic blockers, angiotensin converting enzyme inhibitors, calcium channel blockers, angiotensin II receptor antagonists, beta adrenergic blocking agents, vasodilators, anti-hyperlipidemia drugs and any combination thereof, the use of a drug means for treating the inciting endocrinologic cause such as, but not limited to, a drug means for treating diabetes such as insulin and oral anti-diabetic drugs, a drug means for treating the inciting rheumatologic cause such as, but not limited to, a drug means for treating rheumatoids, the use of a drug means for treating the inciting trauma cause including the decompression of the facial nerve such as diuretics. The Theory of Bell's palsy also provides the injection of local anesthetics onto the facial nerve.

Although various preferred embodiments of this invention have been described, it will be appreciated by those skilled in the art that adaptations and variations may be made without departing from the spirit of the invention and the scope of the claims.

1. The Theory of Bell's palsy based on scientific evidences and proofs comprises:
facial paralysis is preceded by abnormal, altered or decreased sensations of the facial structures;
facial paralysis is preceded by abnormal, altered or decreased activities of the facial structures;
two types of facial paralysis based on two distinct inciting etiologies wherein a first facial paralysis is caused by a first viral, bacterial, spirochetal or fungal infection;
said first facial paralysis is caused by the compression and ischemia of the facial nerve;
two types of facial paralysis based on two distinct etiologies wherein a second facial paralysis is caused by a second vascular etiology associates with cardiovascular diseases
two types of facial paralysis based on two distinct etiologies wherein a second facial paralysis is caused by a second vascular etiology associates with endocrinologic diseases
two types of facial paralysis based on two distinct etiologies wherein a second facial paralysis is caused by a second vascular etiology associates with rheumatolgic diseases;
two types of facial paralysis based on two distinct etiologies wherein a second facial paralysis is caused by a second vascular etiology associates with trauma;
said second facial paralysis is caused by the compression and ischemia of the facial nerve;
said second facial paralysis associates with vascular etiology, in general, is more virulent than said first facial paralysis;
said second facial paralysis associates with vascular etiology, in general, is more severe;
said second facial paralysis associates with vascular etiology, in general, has the tendency towards poorer outcomes than said first facial paralysis;
a mathematic means for defining the duration of the facial paralysis and recovery time from the facial paralysis; and
a process of managing the etiologies of facial paralysis.

2. The Theory of Bell's palsy according to claim 1 wherein said process of managing the etiologies facial paralysis is the use of a drug means for treating the inciting cardiovascular cause.

3. The Theory of Bell's palsy according to claim 1 wherein said process of managing the etiologies of facial paralysis is the use of a drug means for treating hypertension.

4. The Theory of Bell's palsy according to claim 1 wherein said process of managing the etiologies of facial paralysis is the use of a drug means for treating the inciting endocrinologic cause.

5. The Theory of Bell's palsy according to claim 1 wherein said process of managing the etiologies of facial paralysis is the use of a drug means for treating the inciting rheumatologic cause.

6. The Theory of Bell's palsy according to claim 1 wherein said process of managing the etiologies of facial paralysis is the use of a drug means for treating the inciting trauma cause.

7. The Theory of Bell's palsy according to claim 1 wherein said process of managing the etiologies of facial paralysis is the injection of local anesthetics onto the facial nerve;

8. The Theory of Bell's palsy according to claim 1 wherein a mathematic means for defining the duration of the facial paralysis and recovery time from the facial paralysis is an equation Y=f(X)+K.

9. The equation according to claim 8 is Y=cA+cB+K.

10. The Theory of Bell's palsy according to claim 1 wherein a mathematic means for defining the duration of the facial paralysis and recovery time from the facial paralysis is a plurality of graphs and their derivatives.

11. A method of treating the infection, cardiovascular, endocrinologic and rheumatologic diseases causing unilateral and bilateral facial paralysis based on the Theory of Bell's palsy formed by scientific evidences comprising two types of facial paralysis based on two distinct inciting etiologies wherein a first facial paralysis is caused by a first viral, bacterial, spirochetal or fungal infection and wherein a second facial paralysis is caused by a second vascular etiology associates with cardiovascular, endocrinologic and rheumatolgic diseases; wherein said second facial paralysis associates with vascular etiology, in general, is more virulent than said first facial paralysis comprises:
a process of using a drug means for treating the inciting cardiovascular cause;
a process of using drug means for treating hypertension;
a process of using drug means for treating the inciting endocrinologic cause;
a process of using drug means for treating the inciting rheumatologic cause;
a process of using a drug means for treating the inciting trauma cause; and
a process of injecting a local anesthetics onto the facial nerve.

12. The process of using a drug means for treating the inciting trauma cause according to claim 11 includes the process of using a diuretics.

13. A standard of medical treatment of Bell's palsy based on the Theory of Bell's palsy derived from scientific evidences comprising facial paralysis is preceded by the abnormal, altered or decreased sensations and activities of the structures of the face and mouth, two types of facial paralysis based on two distinct inciting etiologies wherein a first facial paralysis is caused by a first viral, bacterial, spirochetal or fungal infection, wherein said first facial paralysis is caused by the compression and ischemia of the facial nerve, wherein two types of facial paralysis based on two distinct etiologies wherein a second facial paralysis is caused by a second vascular etiology associates with cardiovascular, endocrinologic, rheumatolgic diseases or trauma, wherein said second facial paralysis is caused by the compression and ischemia of the facial nerve, wherein said second facial paralysis associates with vascular etiology, in general, is more virulent than said first facial paralysis, said second facial paralysis associates with vascular etiology, in general, is more severe and has the tendency towards poorer outcomes than said first facial paralysis comprises:
a mathematic means for defining the duration of the facial paralysis and recovery time from the facial paralysis;
a process of using a drug means for treating an etiology of facial paralysis; and
a process of injecting a local anesthetics onto the facial nerve.

14. The standard of medical treatment according to claim 13 wherein said process of using a drug means for treating an etiology of facial paralysis is the process of using a drug means for treating the inciting cardiovascular cause.

15. The standard of medical treatment according to claim 13 wherein said process of using a drug means for treating an etiology of facial paralysis is the process of using a drug means for treating hypertension.

16. The standard of medical treatment according to claim 13 wherein said process of using a drug means for treating an etiology of facial paralysis the process of using a drug means for treating the inciting endocrinologic cause.

17. The standard of medical treatment according to claim 13 wherein said process of using a drug means for treating an etiology of facial paralysis is the process of using a drug means for treating inciting rheumatologic cause.

18. The standard of medical treatment according to claim 13 wherein said process of using a drug means for treating an etiology of facial paralysis is the process of using a drug means for treating inciting trauma cause.
